(19) United States
(12) Patent Application Publication
(10) Pub. No.: US 2025/0271317 A1
(43) Pub. Date: Aug. 28, 2025

(54) STACKED SENSOR DEVICE

(71) Applicant: Infineon Technologies AG, Neubiberg (DE)

(72) Inventors: Moritz SCHLAGMANN, München (DE); Matthias EBERL, Taufkirchen (DE); Heiko FRÖHLICH, Radebeul (DE); Stefan HAMPL, Dresden (DE); Thoralf KAUTZSCH, Dresden (DE); Vladislav KOMENKO, Dresden (DE); Andrey KRAVCHENKO, Dresden (DE); Alexander KAUFMANN, Freiberg (DE)

(21) Appl. No.: 19/042,628

(22) Filed: Jan. 31, 2025

(30) Foreign Application Priority Data
Feb. 23, 2024 (DE) 102024201710.8

(51) Int. Cl. G01L 9/00; G01N 27/12; G01N 27/22

(52) U.S. Cl. CPC G01L 9/0042; G01N 27/121; G01N 27/223

(57) ABSTRACT

A stacked sensor device includes a micro electromechanical system, MEMS, pressure sensor including a pressure sensor substrate having a recess formed therein, a flexible MEMS structure covering the recess, thereby forming a hermetic chamber within the pressure sensor substrate, and pressure sensing means for detecting a deflection of the flexible MEMS structure. The stacked sensor device further includes a gas sensor including gas sensing means for detecting a property of an ambient gas, the gas sensor being arranged on a standoff above the pressure sensor, such that a cavity is formed by the pressure sensor, the standoff and the gas sensor, and an opening that couples the cavity to the ambient gas.

### CROSS-REFERENCE TO RELATED APPLICATION

This application claims priority to Germany Patent Application No. 102024201710.8 filed on Feb. 23, 2024, the content of which is incorporated by reference herein in its entirety.

### TECHNICAL FIELD

The present disclosure relates to a stacked sensor device comprising a MEMS pressure sensor and a gas sensor as well as to a method of forming a stacked sensor device.

### BACKGROUND

In recent years, advancements in sensor technology have paved the way for integrated devices that provide enhanced capabilities for monitoring environmental conditions. Until recently, integrated sensors have typically operated in isolation, thus limiting the ability to capture a holistic picture of environmental changes. By combining the strengths of a gas sensor, e.g., a humidity sensor, and a pressure sensor within a single, integrated framework, both an improved accuracy and an expansion of the range of potential applications of gas sensing is achieved. The range of applications include gas analysis, battery monitoring, process monitoring, human comfort measurement, or heating, ventilating, and air conditioning (HVAC) applications. Conventional integrated devices that include a humidity sensor and a pressure sensor typically feature a side-by-side arrangement of the sensing elements on the chip. However, such an arrangement easily clashes with the increasing demand of small-footprint system-in-package solutions. A further disadvantage is the cost-inefficient manner of such a side-by-side arrangement as chip surface is expensive.

### SUMMARY

In some implementations, a stacked sensor device includes a micro electromechanical system, MEMS, pressure sensor that includes a pressure sensor substrate having a recess formed therein, a flexible MEMS structure covering the recess, thereby forming a hermetic chamber within the pressure sensor substrate, and pressure sensing means for detecting a deflection of the flexible MEMS structure. The stacked sensor device in these implementations further include a gas sensor that includes gas sensing means for detecting a property of an ambient gas around the stacked sensor device. The gas sensor is arranged on a standoff above the pressure sensor, such that a cavity is formed by the pressure sensor, the standoff, and the gas sensor. Furthermore, the stacked sensor device has an opening that couples the cavity to the ambient gas.

In some implementations, a method of forming a stacked sensor device includes providing a micro electromechanical system, MEMS, pressure sensor that includes a pressure sensor substrate having a recess formed therein, a flexible MEMS structure covering the recess, thereby forming a hermetic chamber within the pressure sensor substrate, and pressure sensing means for detecting a deflection of the flexible MEMS structure. The method further includes arranging a standoff on a top surface of the MEMS pressure sensor, arranging a gas sensor on a top surface of the standoff facing away from the MEMS pressure sensor, wherein the gas sensor includes gas sensing means for detecting a property of an ambient gas. The method further includes forming an opening, wherein the pressure sensor, the standoff and the gas sensor form a cavity, and the opening couples the cavity to the ambient gas.

Those skilled in the art will recognize additional features and advantages upon reading the following detailed description, and upon viewing the accompanying drawings.

### BRIEF DESCRIPTION OF THE DRAWINGS

The present disclosure is illustrated by way of example, and not by way of limitation, in the figures of the accompanying drawings in which like reference numerals refer to similar or identical elements. The elements of the drawings are not necessarily to scale relative to each other. The features of the various illustrated examples can be combined unless they exclude each other.

FIG. 1 illustrates a cross-sectional view of a first example implementation of a stacked sensor device.

FIG. 2 illustrates a cross-sectional view of a second example implementation of a stacked sensor device.

FIG. 3 illustrates a cross-sectional view of a third example implementation of a stacked sensor device.

FIG. 4 illustrates a cross-sectional view of a fourth example implementation of a stacked sensor device.

FIG. 5 illustrates a top view of an example implementation of a humidity sensor of a stacked sensor device.

### DETAILED DESCRIPTION

Various examples will now be described more fully with reference to the accompanying drawings in which some examples are illustrated. In the figures, the thicknesses of lines, layers and/or regions may be exaggerated for clarity.

Accordingly, while further examples are capable of various modifications and alternative forms, some particular examples thereof are shown in the figures and will subsequently be described in detail. However, this detailed description does not limit further examples to the particular forms described. Further examples may cover all modifications, equivalents, and alternatives falling within the scope of the disclosure. Like numbers refer to like or similar elements throughout the description of the figures, which may be implemented identically or in modified form when compared to one another while providing for the same or a similar functionality.

It will be understood that when an element is referred to as being “connected” or “coupled” to another element, the elements may be directly connected or coupled or via one or more intervening elements. If two elements A and B are combined using an “or”, this is to be understood to disclose all possible combinations, e.g., only A, only B as well as A and B. An alternative wording for the same combinations is “at least one of A and B”. The same applies for combinations of more than 2 elements.

The terminology used herein for the purpose of describing particular examples is not intended to be limiting for further examples. Whenever a singular form such as “a,” “an” and “the” is used and using only a single element is neither explicitly or implicitly defined as being mandatory, further examples may also use plural elements to implement the same functionality. Likewise, when a functionality is subsequently described as being implemented using multiple elements, further examples may implement the same functionality using a single element or processing entity. It will be further understood that the terms “comprises,” “comprising,” “includes” and/or “including,” when used, specify the presence of the stated features, integers, steps, operations, processes, acts, elements and/or components, but do not preclude the presence or addition of one or more other features, integers, steps, operations, processes, acts, elements, components and/or any group thereof.

Unless otherwise defined, all terms (including technical and scientific terms) are used herein in their ordinary meaning of the art to which the examples belong.

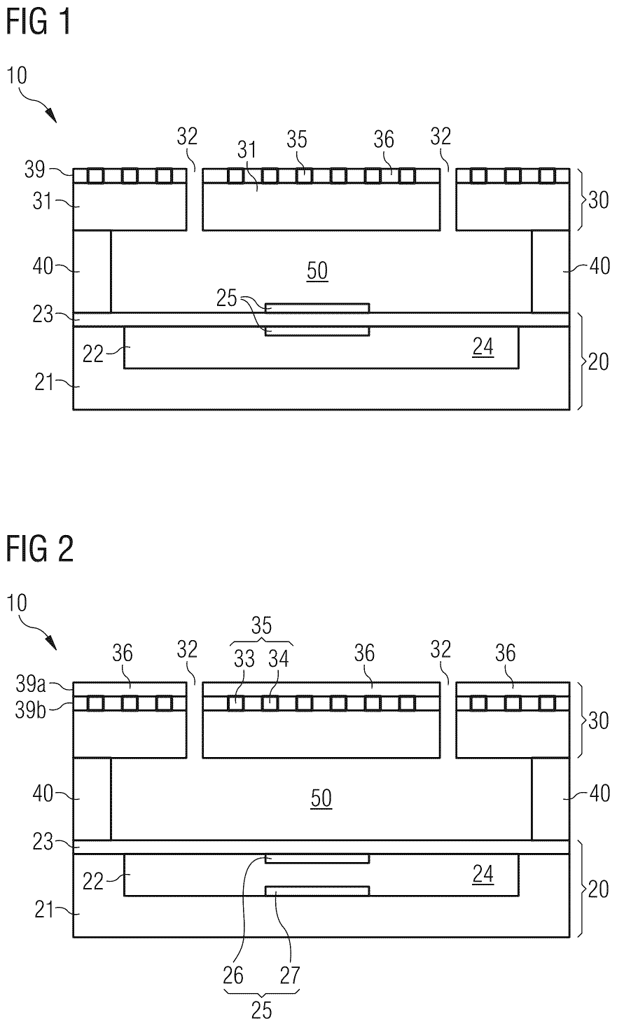

FIG. 1 illustrates a cross-sectional view of a first example implementation of a stacked sensor device 10. The stacked sensor device 10 includes a micro electromechanical system, MEMS, pressure sensor 20 comprising a pressure sensor substrate 21 formed from a semiconductor material, e.g., silicon. A recess 22 is formed within the pressure sensor substrate 21. The pressure sensor 20 further comprises a flexible MEMS structure 23 arranged on a surface of the pressure sensor substrate 21 such that the recess 22 is covered, in particular the recess 22 is completely covered, by the flexible MEMS structure 23. The flexible MEMS structure for example is a diaphragm or a membrane formed from a semiconductor material, e.g., silicon or silicon nitride. Thus, the covered recess 22 forms a hermetic chamber 24 that is sealed from an environment of the pressure sensor 20, in particular from the cavity 50. The pressure sensor 20 further comprises pressure sensing means 25 for detecting a deflection of the flexible MEMS structure 23. For example, the pressure sensing means 25 comprise electrodes arranged on opposite sides of the flexible MEMS structure 23. For example, a capacitance between the electrodes depends on a deflection of the flexible MEMS structure 23. The deflection of the flexible MEMS structure 23 in turn can depend on a pressure difference between a pressure of a vacuum or of a gas within the hermetic chamber 24 and a gas pressure outside the hermetic chamber 24, e.g., a gas pressure of a gas within the cavity 50. The working principle of MEMS pressure sensors is a well-established concept and is not further detailed throughout this disclosure.

The stacked sensor device 10 further comprises one or multiple standoffs 40 that are arranged on a top surface of the pressure sensor 20. For example, the standoffs are arranged on a top layer of the pressure sensor, which can be the flexible MEMS structure 23, for instance, as illustrated. Alternatively, the standoffs 40 can be arranged on surfaces of the pressure sensor substrate 21 that are not covered by the layer comprising the flexible MEMS structure 23. For example, the standoffs 40 are formed from a semiconductor material that can be the same material as a material of the pressure sensor substrate 21, e.g., silicon.

The stacked sensor device 10 further comprise a gas sensor 30 that is arranged on a surface of the standoffs 40 facing away from the pressure sensor 20. In other words, the pressure sensor 20, the standoffs 40 and the humidity sensor 30 from a stacked structure, e.g., the stacked sensor device 10. The humidity sensor comprises a gas sensor substrate 31, which is formed from a semiconductor material that can be the same material as a material of the pressure sensor substrate 21 and/or of the standoffs 40, e.g., silicon. The gas sensor 30 further includes in this example implementation a sensing layer 39 arranged on a top surface of the gas sensor substrate 31, wherein the sensing layer 39 comprises gas sensing means 35 (e.g., a gas sensing circuit), e.g., an electrode structure comprising two electrodes for measuring a capacitance between the electrodes, for instance. For example, the gas sensor 30 is a humidity sensor 30, the gas sensor substrate 31 is a humidity sensor substrate 31, and the gas sensing means 35 are humidity sensing means 35 (e.g., a humidity sensing circuit, or a gas sensing circuit with a humidity sensing circuit). Alternatively, the gas sensor can be a different type such as a temperature sensor or a chemicapacitive sensor for volatile organic compounds, for instance.

Moreover, in this example implementation, a gas sensitive element 36 is arranged within the sensing layer 39 in voids between the humidity sensing means 35. The gas sensitive material 36 can be configured to adsorb or absorb water from the ambient gas and can have a dielectric property that depends on an amount of water adsorbed and/or absorbed by the gas sensitive element 36. For example, the gas sensitive element 36 can be formed from a hygroscopic dielectric material, e.g., a plastic, a polymer such as a polyimide, or an oxide such as silicon oxide, with a dielectric constant that is proportional to an amount of water absorbed by the gas sensitive element 36. Typically, at equilibrium conditions, the amount of moisture present in a hygroscopic material depends on both ambient temperature and ambient water vapor pressure. Therefore, absorption of moisture using the gas sensitive element 36 of the humidity sensor 30 results in an increase in a capacitance measured across a pair of electrodes forming the humidity sensing means 35 placed around the gas sensitive element 36 as described and illustrated. The working principle of capacitive humidity sensors is a well-established concept and is not further detailed throughout this disclosure. Alternatively to a capacitive humidity sensor, the humidity sensor 30 can be configured as a resistive, e.g., piezoresistive, humidity sensor.

The pressure sensor 20, the standoffs 40 and the humidity sensor form a cavity 50. In other words, stacked sensor device 10 comprises a cavity 50 that is delimited by the pressure sensor 20, e.g., by the flexible MEMS structure 23, the standoff 40, and the humidity sensor 30, e.g., by the humidity sensor substrate 31. The stacked sensor device 10 further comprises one or multiple openings 32 that couple the cavity 50 to an ambient gas surrounding the stacked sensor device 10, for instance. The one or multiple openings 32 can be the only coupling between the cavity 50 and the ambient gas. For example, the stacked sensor device 32 comprises one or multiple openings 32 that extend from a top surface of the humidity sensor 30 to the cavity 50 as illustrated. Alternatively or in addition, an opening 32 can extend through the standoff 40. The cavity 50 can be configured to thermally decouple the pressure sensor 20 from the humidity sensor 30. Furthermore, the one or multiple openings 32 each can be dimensioned that gas molecules of an analyte gas, e.g., hydrogen or air, can enter the cavity 50, while larger particles, e.g., contaminants, are prevented from entering the cavity 50. Thus, the forming of a cavity 50 can act as a particle protection for the pressure sensor 20. Moreover, the humidity sensor 30 can act as integrated electromagnetic compatibility (EMC) shielding for the pressure sensor 20.

The stacked sensor device 10 may further comprise active semiconductor components that may be part of an ASIC. The ASIC may be used for acquiring and processing electronic measurement signals from the pressure sensor 20 and the humidity sensor 30 and for generating pressure and humidity signals from the acquired measurement signals.

FIG. 2 illustrates a cross-sectional view of a second example implementation of a stacked sensor device 10. In this implementation, the pressure sensing means 25 (e.g., a pressure sensing circuit) of the pressure sensor include a first electrode 26 arranged at a bottom side of the recess 22 opposite the flexible MEMS structure 23, and a second electrode 27 arranged on a side of the flexible MEMS structure 23 facing the recess 22. In other words, the first and second electrodes are arranged inside the hermetic chamber 24 and opposite each other in a vertical direction with respect to a main plane of extension of the pressure sensor 20. Thus, a capacitance measured across the first and second electrodes 26, 27 can depend on a deflection of the flexible MEMS structure 23 and hence on a gas pressure of gas inside the cavity 50.

Moreover, in this second example implementation, the humidity sensor 30 comprises a capping layer 39a that acts as the gas sensitive element 36. Thus, the capping layer 39a can be at least in portions formed from a hygroscopic dielectric material, e.g., a plastic, a polymer such as a polyimide, or an oxide such as silicon oxide, with a dielectric constant that is proportional to an amount of water absorbed by the capping layer 39a. Underneath the capping layer 39a, e.g., in between the humidity sensor substrate 31 and the capping layer 39a, an electrode layer 39b is arranged comprising a first electrode structure 33 and a second electrode structure 34 as the humidity sensing means 35 (e.g., a humidity sensing circuit, or a gas sensing circuit with a humidity sensing circuit). For example, the first and second electrode structures 33, 34 form an interdigitated electrode structure, across which a capacitance can be measured that depends on an amount of water adsorbed and/or absorbed by the gas sensitive element 36 of the capping layer 39a. The first and second electrode structures 33, 34 can be separated by voids or an insulating material, e.g., silicon or silicon oxide. Furthermore, in between the electrode layer 39b and the capping layer 39a and/or in between the electrode layer and the humidity sensor substrate 31 an additional passivation layer can be arranged, e.g., a thin layer formed from silicon oxide or other suitable materials.

The opening 32 in this example implementation extends from the cavity 50 in a vertical direction through the humidity sensor substrate 31, the electrode layer 39b, and the capping layer 39a as well as through the optional additional passivation layers.

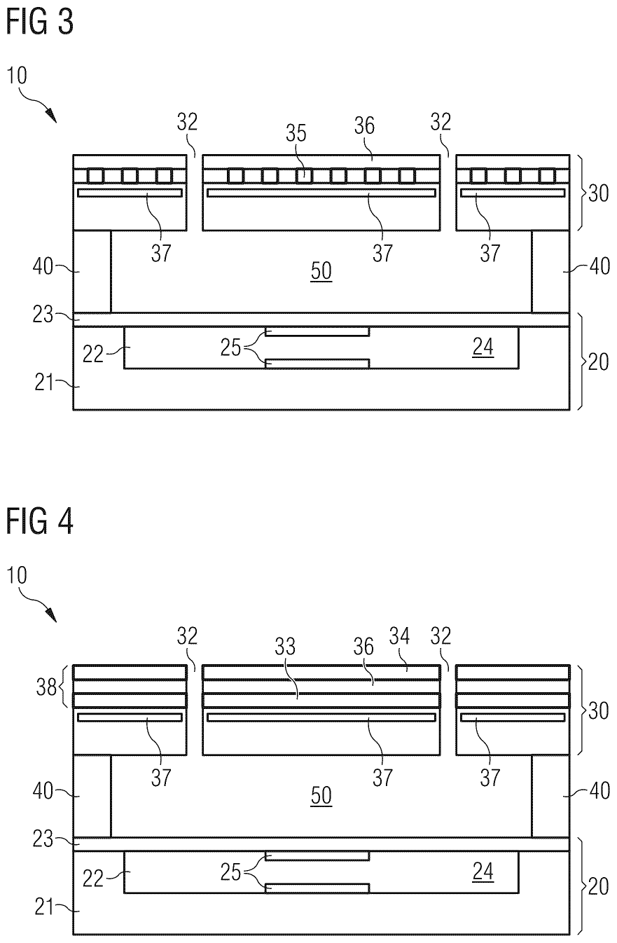

FIG. 3 illustrates a cross-sectional view of a third example implementation of a stacked sensor device 10. In contrast to the second implementation, in this third implementation the stacked sensor device 10 further comprises a heater structure 37 arranged in proximity to the humidity sensing means 35 and to the gas sensitive element 36. For example, the heater structure 37 is arranged within the humidity sensor substrate 31 as illustrated. Alternatively, the heater structure 37 can be arranged in an intermediate layer arranged in between the humidity sensor substrate 31 and a layer comprising the humidity sensing means 35 and/or the gas sensitive element 36. The heater structure 37 can be formed from resistive heating elements, for example. The heater structure 37 is operable to heat the gas sensitive element 36, e.g., for reconditioning purposes. In other words, the heater structure 37 is operable to heat the gas sensitive element 36 to evaporate adsorbed or absorbed water from the gas sensitive element 36. This heating could be performed in between humidity measurements. To this end, the stacked sensor device 10 can further comprise active semiconductor components that may be part of an ASIC. The ASIC may be used for activating, deactivating and controlling the heater structure 37 and for thermalizing the gas sensitive element 36 to a target temperature. The humidity sensor 30 for temperature stabilization can further comprise a thermometer, e.g., a thermistor, arranged in proximity to the gas sensitive element 36.

In implementations, in which the humidity sensor 30 comprises the heater structure 37, the cavity 50 in addition to EMC shielding and particle protection also thermally decouples the humidity sensor 30 from the pressure sensor 20. This enables power-efficient usage of the sensor also under harsh and high-humidity conditions.

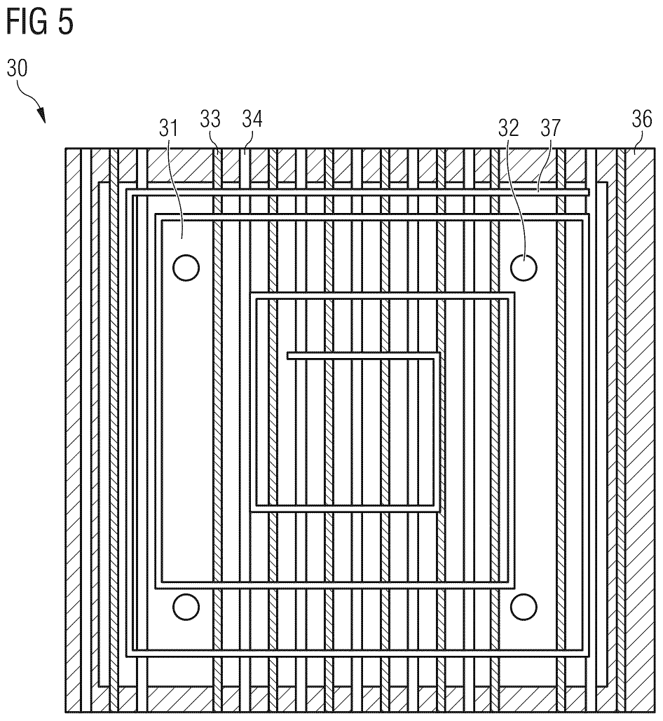

FIG. 4 illustrates a cross-sectional view of a fourth example implementation of a stacked sensor device 10. In contrast to the third implementation, the humidity sensor 30 comprises a stacked structure 38 formed by a first layer comprising the first electrode structure 33, a second layer comprising the gas sensitive element 36, and a third layer comprising the second electrode structure 34, the stacked structure 38 realizing a sandwich structure extending in a perpendicular direction with respect to a main plane of extension of the humidity sensor 30. Optional passivation layers, for instance, can be arranged in between the first, second and third layers of the stacked structure 38 and/or in between the humidity sensor substrate 31 and the stacked structure 38. Furthermore, a capping layer 39a can be arranged on a top surface of the stacked structure 38 facing away from the humidity sensor substrate 31.

FIG. 5 illustrates a top view of an example implementation of a humidity sensor 30 of a stacked sensor device 10. The implementation shows the humidity sensor substrate 31 comprising multiple openings 32 towards the cavity 50, the heater structure 37 arranged within or on a top surface of the humidity sensor substrate 31, and the humidity sensing means 35 formed by a plurality of parallel pairs of first electrode structures 33 and second electrode structures 34. For illustration purposes, the gas-sensitive element 36, for example arranged within a capping layer 39a on a top surface of the electrode layer 38b comprising the first and second electrode structures 33, 34, is merely indicated in the Figure.

FIGS. 1 to 5 show only stacked sensor devices 10 having single sets of a pressure sensor 20 and a humidity sensor 30. However, several sets of stacked sensor devices 10 may be combined to form an array of sets of stacked sensor devices 10. In addition, each stacked sensor devices 10 may comprise more than one pressure sensor 20 and/or more than one humidity sensor 30.

The combination of a particle and EMC protected pressure sensor 20 and humidity sensor 30, the described stacked sensor device 10 brings the advantage of cost-efficient, 3D integration for a system-in-package. With the cavity 50 being arranged in between the optional heater structure 31 and the pressure sensor 20, a power-efficient usage of the sensor even under harsh and high humidity conditions is enabled. The geometry and arrangement of the example pressure and humidity sensing means 25, 35, the heater structure 37 can be adapted to optimize for example sensitivity and/or robustness.

Although specific examples have been illustrated and described herein, it will be appreciated by those of ordinary skill in the art that a variety of alternate and/or equivalent implementations may be substituted for the specific examples shown and described without departing from the scope of the present implementation. This application is intended to cover any adaptations or variations of the specific examples discussed herein. Therefore, it is intended that this implementation be limited only by the claims and the equivalents thereof.

It should be noted that the methods and devices including its preferred implementations as outlined in the present document may be used stand-alone or in combination with the other methods and devices disclosed in this document. In addition, the features outlined in the context of a device are also applicable to a corresponding method, and vice versa. Furthermore, all aspects of the methods and devices outlined in the present document may be arbitrarily combined. In particular, the features of the claims may be combined with one another in an arbitrary manner.

It should be noted that the description and drawings merely illustrate the principles of the proposed methods and systems. Those skilled in the art will be able to implement various arrangements that, although not explicitly described or shown herein, embody the principles of the implementation and are included within its spirit and scope. Furthermore, all examples and implementations outlined in the present document are principally intended expressly to be only for explanatory purposes to help the reader in understanding the principles of the proposed methods and systems. Furthermore, all statements herein providing principles, aspects, and implementations of the implementation, as well as specific examples thereof, are intended to encompass equivalents thereof.

### ASPECTS

In particular, the following aspects are disclosed:

Aspect 1: A stacked sensor device (10), comprising a micro electromechanical system, MEMS, pressure sensor (20) including a pressure sensor substrate (21) having a recess (22) formed therein, a flexible MEMS structure (23) covering the recess (22), thereby forming a hermetic chamber (24) within the pressure sensor substrate (21), and pressure sensing means (25) for detecting a deflection of the flexible MEMS structure (23). The stacked sensor device (10) further comprises a gas sensor (30) including gas sensing means (35) for detecting a property of an ambient gas, the gas sensor (30) being arranged on a standoff (40) above the pressure sensor (20), such that a cavity (50) is formed by the pressure sensor (20), the standoff (40) and the gas sensor (30), and an opening (32) that couples the cavity (50) to the ambient gas.

Aspect 2: The stacked sensor device (10) according to aspect 1, wherein the opening (32) extends from a top surface of the gas sensor (30) to the cavity (50).

Aspect 3: The stacked sensor device (10) according to aspect 1 or 2, wherein the cavity (50) thermally decouples the pressure sensor (20) from the gas sensor (30).

Aspect 4: The stacked sensor device (10) according to one of aspects 1 to 3, wherein the opening (32) is dimensioned to allow gas molecules to enter the cavity (50), and to prevent contaminating particles from entering the cavity (50).

Aspect 5: The stacked sensor device (10) according to one of aspects 1 to 4, wherein the gas sensor (30) comprises a gas sensor substrate (31), and wherein the standoff (40), the pressure sensor substrate (21) and the gas sensor substrate (31) are formed from the same material.

Aspect 6: The stacked sensor device (10) according to one of aspects 1 to 5, wherein the gas sensor (30) is a capacitive or resistive gas sensor.

Aspect 7: The stacked sensor device (10) according to one of aspects 1 to 6, wherein the gas sensing means (35) comprise a first electrode structure (33), a second electrode structure (34) and a gas sensitive element (36), the gas sensitive element (36) being configured to adsorb water from the ambient gas and having a dielectric property that depends on an amount of water adsorbed by the gas sensitive element (36).

Aspect 8: The stacked sensor device (10) according to aspect 7, wherein the gas sensor (30) further comprises a heater structure (37).

Aspect 9: The stacked sensor device (10) according to aspect 8, wherein the heater structure (37) is operable to heat the gas sensitive element (36).

Aspect 10: The stacked sensor device (10) according to aspect 8 or 9, wherein the heater structure (37) is arranged between the cavity (50) and the gas sensitive element (36).

Aspect 11: The stacked sensor device (10) according to one of aspects 7 to 10, wherein a capacitance between the first electrode structure (33) and the second electrode structure (34) depends on the dielectric property of the gas sensitive element (36).

Aspect 12: The stacked sensor device (10) according to one of aspects 7 to 11, wherein the first electrode structure (33), the gas sensitive element (36) and the second electrode structure (34) form a stacked structure (38) extending in a perpendicular direction with respect to a main plane of extension of the gas sensor (30).

Aspect 13: The stacked sensor device (10) according to one of aspects 7 to 11, wherein the first electrode structure (33) and the second electrode structure (34) are arranged in a sensing layer (39) with the gas sensitive element (36) being arranged within the sensing layer (39) in between the first electrode structure (33) and the second electrode structure (34).

Aspect 14: The stacked sensor device (10) according to one of aspects 7 to 11, wherein the first electrode structure (33) and the second electrode structure (34) form interdigitated electrodes with the gas sensitive element (36) arranged in between and/or on the interdigitated electrodes.

Aspect 15: The stacked sensor device (10) according to one of aspects 7 to 14, wherein the gas sensitive element (36) is formed from an oxide, in particular from a silicon oxide, or from a polymer, in particular from a polyimide.

Aspect 16: The stacked sensor device (10) according to one of aspects 7 to 15, wherein the gas sensor (30) further comprises a capping layer (39a) arranged on the first and second electrode structures (33, 34).

Aspect 17: The stacked sensor device (10) according to aspect 16, wherein the capping layer (39a) is configured as the gas sensitive element (36).

Aspect 18: The stacked sensor device (10) according to one of aspects 1 to 17, wherein the flexible MEMS structure (23) is a membrane or a diaphragm.

Aspect 19: The stacked sensor device (10) according to one of aspects 1 to 18, wherein the pressure sensing means (25) of the pressure sensor (20) comprise a first electrode (26) arranged at a bottom side of the recess (22) opposite the flexible MEMS structure (23), and a second electrode (27) arranged on a side of the flexible MEMS structure (23) facing the recess (22).

Aspect 20: The stacked sensor device (10) according to one of aspects 1 to 19, wherein the gas sensor (30) is a humidity sensor having humidity sensing means as the gas sensing means (35), the humidity sensor being configured to detect a humidity level in the ambient gas as the property of the ambient gas.

Aspect 21: A method of forming a stacked sensor device (10), the method comprising: providing a micro electromechanical system, MEMS, pressure sensor (20), the MEMS pressure sensor (20) comprising a pressure sensor substrate (21) having a recess (22) formed therein, a flexible MEMS structure (23) covering the recess (22), thereby forming a hermetic chamber (24) within the pressure sensor substrate (21), and pressure sensing means (25) for detecting a deflection of the flexible MEMS structure (23). The method further comprises arranging a standoff (40) on a top surface of the MEMS pressure sensor (20), arranging a gas sensor (30) on a top surface of the standoff (40) facing away from the MEMS pressure sensor (20), the gas sensor (30) comprising gas sensing means (35) for detecting a property of an ambient gas, and forming an opening (32), wherein the pressure sensor (22), the standoff (40) and the humidity sensor (30) form a cavity (50), and the opening (32) couples the cavity (50) to the ambient gas.

Aspect 22: The method according to claim 21, wherein arranging the gas sensor (30) comprises arranging a humidity sensor on a top surface of the standoff (40).

1. A stacked sensor device, comprising:
a micro-electromechanical system (MEMS) pressure sensor comprising:
a pressure sensor substrate having a recess formed in the pressure sensor substrate;
a flexible MEMS structure covering the recess, thereby forming a hermetic chamber within the pressure sensor substrate; and
a pressure sensing circuit for detecting a deflection of the flexible MEMS structure;
a gas sensor comprising a gas sensing circuit for detecting a property of an ambient gas, the gas sensor being arranged on a standoff above the MEMS pressure sensor, such that a cavity is formed by the MEMS pressure sensor, the standoff, and the gas sensor; and
an opening that couples the cavity to the ambient gas.

2. The stacked sensor device according to claim 1, wherein the gas sensor is a humidity sensor having a humidity sensing circuit as the gas sensing circuit, the humidity sensor being configured to detect a humidity level in the ambient gas as the property of the ambient gas.

3. The stacked sensor device according to claim 1, wherein the opening extends from a top surface of the gas sensor to the cavity.

4. The stacked sensor device according to claim 1, wherein the cavity thermally decouples the MEMS pressure sensor from the gas sensor.

5. The stacked sensor device according to claim 1, wherein the opening is dimensioned to allow gas molecules to enter the cavity, and to prevent contaminating particles from entering the cavity.

6. The stacked sensor device according to claim 1, wherein the gas sensor comprises a gas sensor substrate, and
wherein the standoff, the pressure sensor substrate, and the gas sensor substrate are formed from the same material.

7. The stacked sensor device according to claim 1, wherein the gas sensor is a capacitive gas sensor or a resistive gas sensor.

8. The stacked sensor device according to claim 1, wherein the gas sensing circuit comprise a first electrode structure, a second electrode structure, and a gas sensitive element, the gas sensitive element being configured to adsorb water from the ambient gas and having a dielectric property that depends on an amount of water adsorbed by the gas sensitive element.

9. The stacked sensor device according to claim 8, wherein the gas sensor further comprises a heater structure.

10. The stacked sensor device according to claim 9, wherein the heater structure is operable to heat the gas sensitive element.

11. The stacked sensor device according to claim 9, wherein the heater structure is arranged between the cavity and the gas sensitive element.

12. The stacked sensor device according to claim 8, wherein a capacitance between the first electrode structure and the second electrode structure depends on the dielectric property of the gas sensitive element.

13. The stacked sensor device according to claim 8, wherein the first electrode structure, the gas sensitive element, and the second electrode structure form a stacked structure extending in a perpendicular direction with respect to a main plane of extension of the gas sensor.

14. The stacked sensor device according to claim 8, wherein the first electrode structure and the second electrode structure are arranged in a sensing layer, with the gas sensitive element being arranged within the sensing layer in between the first electrode structure and the second electrode structure.

15. The stacked sensor device according to claim 8, wherein the first electrode structure and the second electrode structure form interdigitated electrodes, with the gas sensitive element being arranged in between or on the interdigitated electrodes.

16. The stacked sensor device according to claim 8, wherein the gas sensitive element is formed from an oxide or from a polymer.

17. The stacked sensor device according to claim 8, wherein the gas sensor further comprises a capping layer arranged on the first electrode structure and second electrode structure.

18. The stacked sensor device according to claim 17, wherein the capping layer is configured as the gas sensitive element.

19. The stacked sensor device according to claim 1, wherein the flexible MEMS structure is a membrane or a diaphragm.

20. The stacked sensor device according to claim 1, wherein the pressure sensing circuit of the MEMS pressure sensor comprises:
a first electrode arranged at a bottom side of the recess opposite the flexible MEMS structure; and
a second electrode arranged on a side of the flexible MEMS structure facing the recess.

21. A method of forming a stacked sensor device, the method comprising:
providing a micro-electromechanical system (MEMS) pressure sensor, the MEMS pressure sensor comprising:
a pressure sensor substrate having a recess formed in the pressure sensor substrate;
a flexible MEMS structure covering the recess, thereby forming a hermetic chamber within the pressure sensor substrate; and
a pressure sensing circuit for detecting a deflection of the flexible MEMS structure;
arranging a standoff on a top surface of the MEMS pressure sensor;
arranging a gas sensor on a top surface of the standoff facing away from the MEMS pressure sensor, the gas sensor comprising gas sensing circuit for detecting a property of an ambient gas; and
forming an opening,
wherein the MEMS pressure sensor, the standoff, and the gas sensor form a cavity, and
wherein the opening couples the cavity to the ambient gas.

22. The method according to claim 21, wherein arranging the gas sensor comprises arranging a humidity sensor on the top surface of the standoff.
